(19) United States
(12) Patent Application Publication
(10) Pub. No.: US 2020/0275912 A1
(43) Pub. Date: Sep. 3, 2020

(54) HUMAN TISSUE QUASI-ELASTIC COEFFICIENT AND ELASTICITY MEASUREMENT METHODS AND DEVICE

(71) Applicant: Xiliang Nie, Elk Grove, CA (US)

(72) Inventor: Xiliang Nie, Elk Grove, CA (US)

(21) Appl. No.: 16/647,559

(22) PCT Filed: Nov. 15, 2018
(86) PCT No.: PCT/CN2018/115524

§ 371 Date: Mar. 16, 2020

(30) Foreign Application Priority Data
Sep. 18, 2017 (CN) 201710842214.7

(51) Int. Cl. A61B 8/08; A61B 8/00; G16H 50/30; G16H 30/40; G16H 50/50; G16H 50/20

(52) U.S. Cl. CPC A61B 8/485; A61B 8/5223; A61B 8/085; A61B 8/4483; A61B 8/565; A61B 8/483; A61B 8/4472; G16H 50/30; G16H 30/40; G16H 50/50; G16H 50/20; A61B 8/4433

(57) ABSTRACT

A human tissue quasi-elastic coefficient measurement method, wherein a simplex optimization method is used to determine human tissue quasi-elastic coefficients, and nine elastic coefficients in three dimensions are reduced to three quasi-elastic coefficients (for two-dimensional ultrasound patient treatment planning or probing, four elastic coefficients are reduced to two quasi-elastic coefficients), thereby saving calculation time. Also provided is a human tissue quasi-elasticity measurement method based on the human tissue quasi-elastic coefficient measurement method, wherein a secondary imaging method is used to measure relative changes in human tissue quasi-elastic coefficients, thereby improving defects in elasticity imaging devices which are dependent on ultrasound imaging accuracy. Changes in human tissue structures may be accurately determined by measuring the relative changes. Also provided is an ultrasound probe or probing device matched with the human tissue quasi-elastic coefficient measurement, which obviates the need for expensive imaging devices, thereby lowering costs.

### TECHNICAL FIELD

The present invention relates to the field of diseases diagnose, and more particularly to a human body tissue quasi-elasticity and quasi-elasticity coefficients or image optic density changes as a clinic disease diagnostic standard and the method of measurement. The present invention also provides a possible patient treatment plan based on ultrasound images which we can do plan every day, people can accurately treat patient with the tumor size change with the treatment time.

### BACKGROUND TECHNIQUE

There are absolute elastic constant determined by the elastic body tissue imager (e.g., GE, or Mindray elastography), but such elastic image analyzer (elastography) generally require expensive, high precision image forming apparatus. It is not clinically accepted by radiation oncologists since they are always identified the calcification (or benign) area and high density texture tissue as cancer tumor and no market at all.

current elasticity coefficient method is generally used to calculate the nine elastic coefficients for 3D system or four elastic coefficients of 2D system, the calculation time is longer, and because the absolute elasticity of human tissue is a difficult task to determine, even if you can get absolute elasticity coefficients, it is not easy to distinguish between normal tissue and diseased tissue elastic coefficient difference (Such as human calcification or benign or even the high density texture tissue area are very similar to the density of the tumor).

It can be seen that the above-mentioned conventional method and apparatus for measuring the absolute elasticity coefficients are inconvenience and it is not precisely distinguish the tumor and the normal tissue, and an urgent needing for theoretical breakthrough to solve this problem is emerging.

### SUMMARY

The present invention discloses a human body tissue determination of quasi-elastic coefficient of elasticity using the simplex optimization method to determine human tissues quasi-elastic coefficient, and the three-dimensional nine elastic coefficients reduced to three quasi-elastic coefficients (like for two-dimensional ultrasound patient treatment plan or detection, the four elastic coefficients are reduced to two quasi-elastic coefficients), saving the calculation time; the present invention also provides a method based on quasi-elastic tissue of the method of determination of elasticity. Two times image is suggested to consider the difference at each pixel as a standard to identify the diseases other than the normal tissue. This will eliminate the problem of the absolute elastic coefficients determination of the tumor (such as in elastography), by considering the relative change, we can use the changing amount to obviously distinguish the disease and normal tissue since the disease always have large change in comparing to normal tissue; Further, the present invention also provides ultrasonic transducer, without the need for expensive imaging computer system, make it lower prices, This patented transducer with the color-marking disease area can be accept by the public with easy using, Suitable for public promotion of the application.

### DESCRIPTION OF THE DRAWINGS

The present invention is merely an overview of the technical solution, and the present invention will be described in further detail with reference to the accompanying drawings and specific embodiments in order to provide a clearer understanding of the technical means of the present invention.

FIG. 1 is a schematic structure of an ultrasonic transducer according to the present invention; we move echo wave recording timer and storage in the computer system of the traditional ultrasound system to the transducer so that end-users don't need image computer to save the end-user's cost. Also it is much more convenient for the diagnostic software update to most current disease identify algorithm since we just have one unique supercomputer diagnostic center worldwide and also save the end-users cost. We introduce 3D image system and color-marking disease area embedded into traditional ultrasound image so that the public end-users can use the transducer at home just under the guide of the manual

FIG. 2 is a block diagram of ultrasonic detection apparatus of the present invention;

FIG. 3 is a schematic view of the diagnostic accuracy of the present invention since we introduce the digital change concept in diagnosing that we can actually mark the disease at pixel level which is about 1 mm×1 mm×1 mm at current ultrasound technology. It is very likely to improve the resolution to couple of μm if we improve echo wave recording timer and piezoelectric ceramics resolutions.

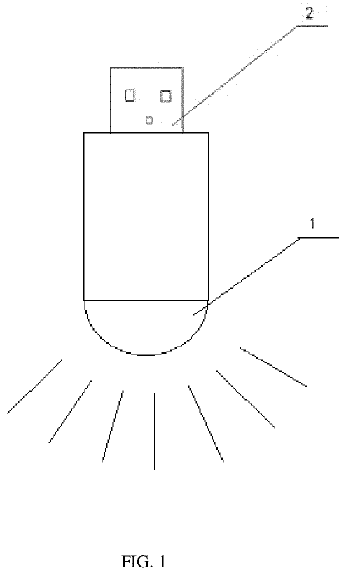

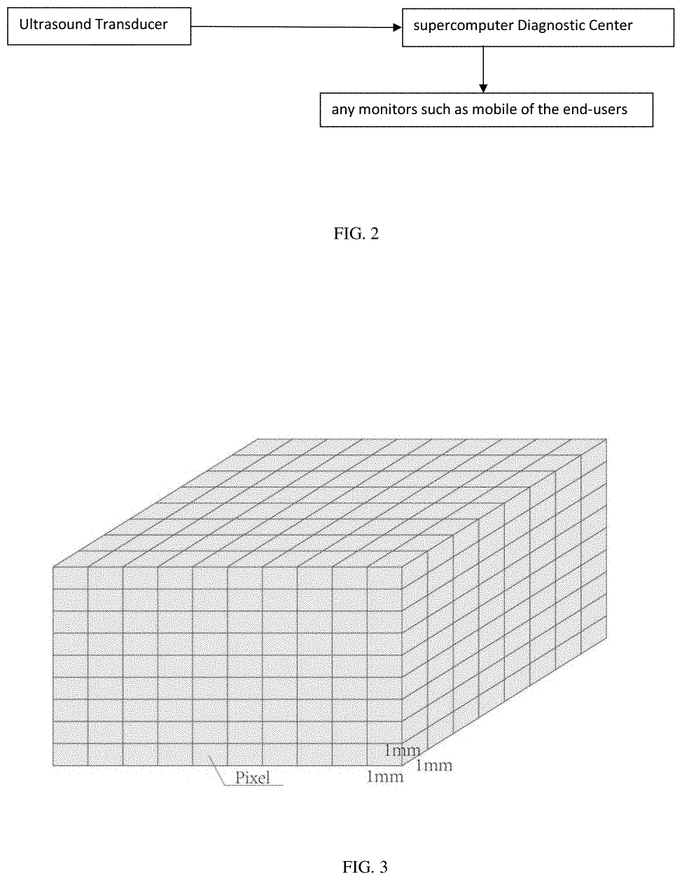

### THE CONTENTS OF THE INVENTION

The first technical problem to be solved by the present invention is to provide a human tissue quasi-coefficient of elasticity, so as to save computing time; to overcome the disadvantages of the long time to calculate the absolute-elastic coefficient. In order to solve the above-mentioned technical problems, the present invention adopts the following technical scheme:

The present invention provides a human body tissue quasi-elastic coefficient of the elasticity, based on the following equation (1) to define the main strain in x, y, and z axial. As by using simplex optimization method to find out the final result of the quasi-elastic coefficients of Eₓₓ, E_{yy} and E_{zz} as describing in the following:

T=EₓₓΔX+E_{yy}ΔY+E_{zz}ΔZ (1)

Equation (1): T is quasi-strain of the human tissue. Eₓₓ, E_{yy}, E_{zz} are three major human tissue elastic coefficients (we called them quasi-elastic coefficients since they are actually not the real physic definition of the elastic coefficients), ΔX, ΔY, ΔZ are the measured image displacement under the pressure;

Specifically:

(1) a first set of assumed elastic coefficients of elasticity Eₓₓ₀, E_{yy}, E_{zz} as input points, ΔX, ΔY, ΔZ are pressure displacements from the same image optic density point obtained by the three-dimensional ultrasonic probe, we calculate quasi-strain as in Eq. (1);

(2) According to simplex optimization method to adjust Eₓₓ, E_{yy} and E_{zz} We can calculate another new quasi-strain;

(3) Continue to do so until the Tₙ−Tₙ₋₁ goes to about zero, we get a human tissue quasi-value of the elasticity.

As further improved, the three-dimensional ultrasonic image (Or two-dimensional) The minimum detection area is at a pixel and current dividing unit is about 1 mm×1 mm×1 mm (For two-dimensional 1 mm×1 mm) Or lower if we improve the timer and piezoelectric ceramics resolution much fine.

Further, we introduce two-times imaging method (the time period is depending on the disease's characteristic such as fast-changing disease would be in couple of hours, the principle should be with the least time period that we wouldn't see obvious change for normal tissue in this time period), which used the difference of two-times images' quantities (such as current quasi-elastic coefficient ΔE or the image optic density ΔI) at the same pixel as criterion to identify the disease and normal tissue since disease has always large variation quantity in comparing to normal tissue. This is an axiom but I introduce this axiom as a clinic standard and patented first to precisely identify disease other than the normal tissue to overcome the existing problem of uncertainty (we can't distinguish between calcification (benign) area even high density normal tissue and tumor) of the absolute elasticity coefficient determination device (such as elastography)

The present invention further provides an ultrasonic transducer adapted to obtain a tissue image density and transmit to the unique supercomputer diagnostic center via an output port such as USB or WIFI.

Clinically the ultrasound devices need to be operated in the hospital by well-trained technicians. We invented new ultrasound transducer as following:

An ultrasonic transducer adapted to obtain a 3D tissue image density and transmit to unique supercomputer diagnostic center via an output port such as USB or WIFI;

A new set of analysis software of the change of the quasi-elasticity of human tissue image or image optic density to accurately distinguish between normal tissue and disease area, We also suggest to use color to mark the different change region to obviously identify the disease area even by public end-users without any medical knowledge the accuracy can be achieved even more than the most advanced the tumor detectors (CT and MRI, we are not say we overcome the physical resolution of the CT and MRI. But our introduction of the change criteria to make the resolution to a pixel-point level) since we introduced digital change of ΔE or ΔI, thus we overcome arbitrary understanding of the disease applying to the most advanced tumor detectors due to traditional radiologist eye viewing image.

We first introduce a pixel identification model to distinguish the disease area other than the normal tissue so that we can make our diagnostic resolution to about 1 mm×1 mm×1 mm for 3D or 1 mm×1 mm for 2D in current ultrasound techniques or even much fine after we improve the timer and piezoelectric ceramics resolution.

This patent's introduction of two-times image change to identify the disease area other than the normal tissue area will automatically reduce some systemic errors of the ultrasound system as we knew that it is a mathematic axiom that the systemic errors would be eliminate from the difference of the same system subtraction. Furthermore our system make it possible to cure the disease in very early stage (there is no any harmful or painful influence to human being) by using Biopsy to remove the disease area under our system's guide since we can see both the disease location and the track of the Biopsy needle.

We have introduced two-times imaging concept to use the change to identify the disease area other than the normal tissue to standardize disease area identification criterion. That is an axiom to be used either by Western medicine or Chinese medicine clinically daily since the starting of the medicine so that we can use AI to precisely diagnose the disease as small as 1 mm×1 mm×1 mm for 3D or 1 mm×1 mm for 2D without any radiologist's clinic experiences. We also introduce ultrasound transducer without the image computer system to save the cost of end-users to make it possible that the disease diagnosing maybe go to home with color-marking of the disease area embedded into original traditional ultrasound image so that both professionals and public end-users liking the system, also we save lots of the money to the end-users since we design an unique supercomputer diagnostic center to handle the end-users' raw data. This is a brave and revolutionary change in the medical field. We also propose to use the Biopsy to remove the disease area in the very early stage. This may make the diseases' pain getting away from human being. We would think this will be a big breakthrough in the medical field.

In summary, the present invention introduce quasi-elastic coefficients of 3 main axial Eₓₓ, E_{yy}, and E_{zz} for 3D image or 2 main axial Eₓₓ, E_{yy}, for 2D image by using simplex optimization technique to short the computer time. Furthermore we also introduced two-times imaging method (the time period is depending on the disease's characteristic such as fast-changing disease would be in couple of hours, the principle should be with the least time period that we wouldn't see obvious change for normal tissue in this time period), which used the pixel difference of two-times images (such as current quasi-elastic coefficients ΔE or the image optic density ΔI) at the same pixel as criterion to identify the disease other than normal tissue since disease has always large change quantity in comparing to normal tissue. This is an axiom. We also introduce a family-friend portable ultrasound 3D transducer to save end-users cost with utilizing the current fast-developed WIFI with cloud concept to give the diagnostic results with very fine resolution (currently in about 1 mm×1 mm×1 mm and further improvement to couple of μm level possible if we could improve the echo wave recording timer and piezoelectric ceramics resolutions) with color-marking of the disease area.

As described above, only the preferred embodiments of the present invention are not intended to be limiting of the present invention claims, and those skilled in the art will make some simple modifications, equivalent variations or modifications in the technical aspects disclosed herein, Within the scope of this invention, in addition, the patient treatment plan based on the principles of the present invention should fall within the scope of the present invention.

1. A human tissue method of determining the quasi-elasticity coefficients, this is the basic principle of this patent protection. Basing on the following Eq. (1) using the simplex optimization method to determine human tissues quasi-elasticity:
T=EₓₓΔX+E_{YY}ΔY+E_{ZZ}ΔZ (1)
In Eq. (1): T is human tissue quasi-tensor of the quasi-elastic coefficients we defined in this patent Eₓₓ, E_{yy} and E_{zz} are three major human tissue elastic coefficients, ΔX, ΔY, and ΔZ are pressure displacement;
Specifically:
(1) a first set of assumed elastic coefficients of elasticity Eₓₓ₀, E_{yy0}, E_{zz0} as input points, ΔX, ΔY, ΔZ are pressure displacements of the same image density obtained by the three-dimensional ultrasonic probe, then we calculate quasi-strain;
(2) According to simplex optimization method to adjust Eₓₓ, E_{yy} and E_{zz} We can calculate another new quasi-strain;
(3) Continue to do so until the Tₙ−Tₙ₋₁ goes to about zero, we get a human tissue quasi-value of the elasticity.

2. According to claim 1, the minimum division of the imaging identified region of the three-dimensional ultrasonic image for tumor is about 1 mm×1 mm×1 mm. That improved current radiologists eye determined tumor size of 0.5 cm to about 1000 times less, which is unique for this patent.

3. According to claim 1, We introduce two-times imaging method (the time period is depending on the disease's characteristic such as fast-changing disease would be in couple of hours, the principle should be with the least time period that we wouldn't see obvious change for normal tissue in this time), which used the pixel difference of two-times images (such as current quasi-elastic coefficients ΔE or the image optic density ΔI) at the same pixel as criterion to identify the disease and normal tissue since disease has always large change quantity in comparing to normal tissue. This is an axiom but I introduce this axiom as a clinic judgment criterion and patent first to precisely identify disease. All methods of using two times imaging method of the present invention for patient pathology change belongs to the scope of this patent protection.

4. Any treatment plan based on claims (1), should be protected with this patent

5. A special ultrasound transducer device without traditional image computer system as illustrated in this patent that it has USB output or WIFI output design for the end-users is protected by this patent.

6. Any production based on current claim (1), are protected by this patent.

7. An ultrasonic diagnostic apparatus, comprising:
an ultrasound transducer with USB or WIFI output including the software based on claims (1), (2), and (3) and the cloud-like supercomputer diagnostic center to analyze the raw data from the end-user's patented transducer and transfer the color-like disease image info embedded in the original traditional ultrasound image with the statistical data files for both professionals and end-users. All above mentioned are in the scope of this patent and protected.

8. According to claim 7, An ultrasonic transducer adapted to obtain a tissue image density and transmit to the unique supercomputer diagnostic center via an output port such as USB or WIFI. This concept is protected by this patent

9. According to claim 1, A new set of analytic software taking into account of the changes of the image at pixel to accurately distinguish between normal tissue and diseases, the accuracy of the distinction can be achieved even more accurate than the most advanced diseases detectors, thus overcoming the most advanced tumor detection the physical radiologist eyes' limit of the reading images. Those kinds of software are protected by this patent.

10. A patient treatment plan according to claim (1), is also within the scope of this patent.
